(19) United States
(12) Patent Application Publication
(10) Pub. No.: US 2023/0270418 A1
(43) Pub. Date: Aug. 31, 2023

(54) ULTRASOUND CATHETER

(71) Applicant: TERUMO KABUSHIKI KAISHA, Tokyo (JP)

(72) Inventors: Hiroyuki ISHIHARA, Tokyo (JP); Katsuhiko SHIMIZU, Fujinomiya-shi (JP); Yasukazu SAKAMOTO, Hiratsuka-shi (JP); Shunsuke YOSHIZAWA, Ebina-shi (JP); Yuki JINGU, Hadano-shi (JP)

(73) Assignee: TERUMO KABUSHIKI KAISHA, Tokyo (JP)

(21) Appl. No.: 18/174,877

(22) Filed: Feb. 27, 2023

(30) Foreign Application Priority Data
Feb. 28, 2022 (JP) 2022-029344

(51) Int. Cl. A61B 8/08; A61B 8/12; A61B 8/00

(52) U.S. Cl. CPC A61B 8/5276; A61B 8/12; A61B 8/4461

(57) ABSTRACT

An ultrasound catheter capable of reducing noise in an ultrasound image by limiting reflection of ultrasound oscillated from a distal end side surface of a transducer. The ultrasound catheter includes: an elongated sheath; an ultrasound transducer; a shaft that supports the ultrasound transducer; and a cap that seals an opening portion of the sheath and has an X-ray opaque property. The cap includes an extension portion including a proximal portion facing the ultrasound transducer and an outer circumferential surface facing an inner circumferential surface of the sheath. The proximal portion includes an ultrasound scattering surface that scatters ultrasound from the ultrasound transducer. The outer circumferential surface includes a contact portion that is in contact with the inner circumferential surface of the sheath in a liquid-tight manner. The ultrasound scattering surface intersects with a virtual plane obtained by enlarging the ultrasound oscillation surface in an in-plane direction.

### CROSS-REFERENCES TO RELATED APPLICATIONS

This application claims priority to Japanese Application No. 2022-029344 filed on Feb. 28, 2022, the entire content of which is incorporated herein by reference.

### TECHNOLOGICAL FIELD

The present disclosure generally relates to an ultrasound catheter that acquires an image by insertion into a lumen of a heart or a blood vessel.

### BACKGROUND DISCUSSION

When a target lesion is examined from a heart, a blood vessel, or the like, an ultrasound catheter that is inserted into a lumen of a living body and acquires an image by using ultrasound. The ultrasound catheter includes a transducer that transmits and receives ultrasound, a shaft that rotates the transducer, and a sheath that rotatably accommodates the transducer and the shaft. The transducer is rotationally driven by the shaft in the sheath so as to transmit and receive the ultrasound and thus acquire an image in a living body.

The transducer faces a direction orthogonal to a central axis of the sheath or a direction slightly inclined from the direction in a closed space of a sheath distal portion. Examples of such an ultrasound catheter include, for example, an ultrasound catheter disclosed in International Patent Publication No. WO2020/196337A.

Since the image is acquired by the ultrasound catheter, when the ultrasound is oscillated from the transducer, the ultrasound is oscillated not only to the direction that the transducer faces but also from a distal end side surface of the transducer. Therefore, the ultrasound is partially directed to a distal end side of the sheath and reflected, and ring-shaped noise appears on an ultrasound image. The ring-shaped noise changes in diameter by moving the transducer in an axial direction of the sheath, thereby inhibiting observation on a biological tissue.

### SUMMARY

An ultrasound catheter is disclosed, which is capable of reducing noise in an ultrasound image by limiting reflection of ultrasound oscillated from a distal end side surface of a transducer.

An ultrasound catheter includes: an elongated sheath having an opening portion at a distal end; an ultrasound transducer accommodated in the sheath; a shaft holding the ultrasound transducer in the sheath; and a cap sealing the opening portion of the sheath and including an X-ray opaque portion having an X-ray opaque property. The ultrasound transducer includes an ultrasound oscillation surface that is substantially parallel to a central axis of the sheath or inclined at an angle of 15 degrees or less with respect to the central axis of the sheath. The cap includes an extension portion including a proximal portion extending from the opening portion into the sheath and facing the ultrasound transducer and an outer circumferential surface facing an inner circumferential surface of the sheath. The proximal portion has an ultrasound scattering surface that scatters ultrasound directed from the ultrasound transducer toward the proximal portion. The outer circumferential surface includes a contact portion that is in contact with the inner circumferential surface of the sheath in a liquid-tight manner. The ultrasound scattering surface intersects with a virtual plane obtained by enlarging the ultrasound oscillation surface in an in-plane direction, and is inclined with respect to a plane perpendicular to the central axis of the sheath.

In the ultrasound catheter configured as described above, the opening portion of the sheath is reliably sealed with the cap having the contrast-enhanced property, and noise generated by reflection of ultrasound propagating from the ultrasound transducer toward the cap to an ultrasound transducer side can be reduced.

The ultrasound scattering surface may be formed on a surface of a substantially conical portion that is convex from the contact portion toward a proximal end side and has a vertex disposed on the central axis of the sheath. Accordingly, the ultrasound from the ultrasound transducer can be reliably reflected toward a side other than the ultrasound transducer side.

The substantially conical portion may have a substantially cone shape. Accordingly, a scattering direction of the ultrasound reflected by the ultrasound scattering surface is constant, and a noise reduction effect can be enhanced.

The substantially conical portion may be formed such that the ultrasound scattering surface has an inclination angle of 35 degrees or larger with respect to the plane perpendicular to the central axis of the sheath. Accordingly, the noise can be further reduced.

The ultrasound oscillation surface may be disposed at a separate position radially outward from the central axis of the sheath. Accordingly, since the vertex of the substantially conical portion is not located on the virtual plane extending from a distal surface of the ultrasound transducer in parallel to the ultrasound oscillation surface, the ultrasound propagating from the ultrasound transducer toward the cap can be more reliably scattered by the ultrasound scattering surface, and the noise can be reduced.

The ultrasound scattering surface may be formed on a surface of a substantially conical portion that is concave in a direction away from the ultrasound transducer and has a vertex disposed on the central axis of the sheath. Accordingly, the ultrasound from the ultrasound transducer can be reliably reflected toward a side other than the ultrasound transducer side.

The cap may include, at a distal end of the extension portion, a distal end protruding portion that protrudes from the opening portion toward a distal end side and has an outer diameter substantially the same as an outer diameter of the sheath, and the contrast-enhanced portion may be constituted by a portion including the distal end protruding portion and the extension portion. Accordingly, a size of the contrast-enhanced portion can be increased, and the contrast-enhanced property can be improved.

The distal end protruding portion may have a dome shape that is convex toward the distal end side. Accordingly, it is possible to prevent a biological tissue from being damaged by the shape of the distal end.

The ultrasound transducer may include an ultrasound attenuation member covering at least a portion which faces the proximal portion of the cap of a side surface substantially perpendicular to the ultrasound oscillation surface. Accordingly, the ultrasound propagating from the ultrasound transducer toward the cap can be further scattered, and the noise can be further reduced.

The ultrasound transducer may move substantially along the central axis of the sheath by the shaft. Accordingly, it is possible to acquire a cross-sectional image along a running direction of a blood vessel in a state in which the noise caused by the ultrasound reflected by the cap is reduced.

An ultrasound catheter is disclosed, which includes: an elongated sheath having an opening portion at a distal end; an ultrasound transducer accommodated in the sheath; a cap sealing the opening portion of the sheath and including an X-ray opaque portion having an X-ray opaque property; and the cap includes an extension portion including a proximal portion extending from the opening portion into the sheath and facing the ultrasound transducer and an outer circumferential surface facing an inner circumferential surface of the sheath, the proximal portion of the cap including an ultrasound scattering surface that scatters ultrasound directed from the ultrasound transducer toward the proximal portion, and the outer circumferential surface of the cap includes a contact portion that is in contact with the inner circumferential surface of the sheath.

An ultrasound catheter is disclosed, which includes: an elongated sheath having an opening portion at a distal end of the sheath; a transducer unit having a plurality of ultrasound transducers, the transducer unit being accommodated in the sheath; a shaft supporting the transducer unit in the sheath; a cap sealing the opening portion of the sheath and including an X-ray opaque portion having an X-ray opaque property; the cap includes an extension portion including a proximal portion extending from the opening portion into the sheath and facing the ultrasound transducer and an outer circumferential surface facing an inner circumferential surface of the sheath, the proximal portion of the cap including an ultrasound scattering surface that scatters ultrasound directed from the ultrasound transducer toward the proximal portion, and the outer circumferential surface of the cap includes a contact portion that is in contact with the inner circumferential surface of the sheath; and wherein the ultrasound scattering surface intersects with a virtual plane obtained by enlarging the ultrasound oscillation surface in an in-plane direction, and is inclined with respect to a plane perpendicular to the central axis of the sheath.

### BRIEF DESCRIPTION OF THE DRAWINGS

FIG. 1 is a front view showing an ultrasound catheter.

FIG. 2 is an enlarged cross-sectional plan view of the vicinity of a distal portion of the ultrasound catheter.

FIG. 3 is an enlarged cross-sectional front view of the vicinity of the distal portion of the ultrasound catheter.

FIG. 4 is a front view of a cap.

FIGS. 5A to 5C are images of a biological tissue acquired by the ultrasound catheter, in which FIG. 5A is an image obtained when a cap having no ultrasound scattering surface is used, FIG. 5B is an image obtained when a cap having the ultrasound scattering surface is used, and FIG. 5C is an image obtained when the cap having the ultrasound scattering surface is used and an ultrasound attenuation member is provided on an ultrasound transducer.

FIG. 6 is a schematic view showing an example of use of an ultrasound catheter system according to the present embodiment.

FIG. 7A to 7G are front views of modifications and comparative examples of the cap.

FIG. 8 is a front view of a cap according to another modification.

FIG. 9 is an enlarged cross-sectional front view of the vicinity of a distal portion of an ultrasound catheter having transducers according to a first modification.

FIG. 10 is an enlarged cross-sectional front view of the vicinity of a distal portion of an ultrasound catheter having transducers according to a second modification.

### DETAILED DESCRIPTION

Set forth below with reference to the accompanying drawings is a detailed description of embodiments of an ultrasound catheter that acquires an image by insertion into a lumen of a heart or a blood vessel. Note that since embodiments described below are preferred specific examples of the present disclosure, although various technically preferable limitations are given, the scope of the present disclosure is not limited to the embodiments unless otherwise specified in the following descriptions. Dimensional ratios in the drawings may be exaggerated and different from actual ratios for convenience of description. Further, in the present description, a side to be inserted into a living body is referred to as a “distal end side”, and a side to be operated is referred to as a “proximal end side”.

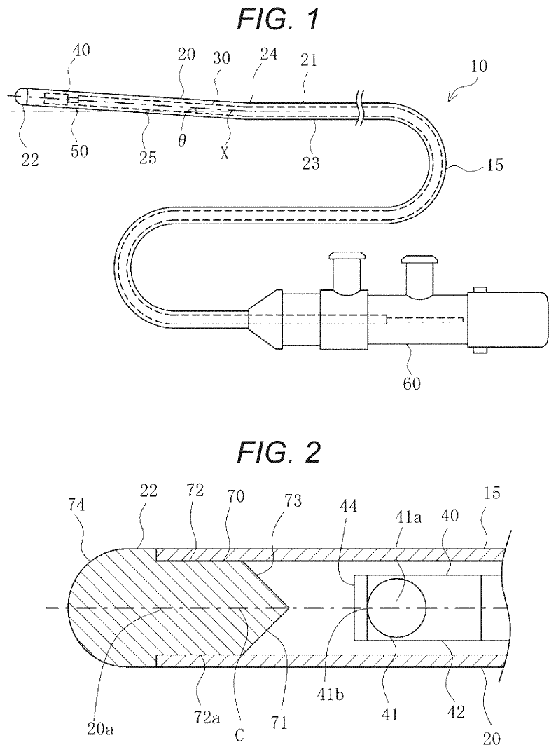

As shown in FIG. 1, an ultrasound catheter 10 according to an embodiment includes a housing 60 at a proximal portion of an elongated sheath 15. The sheath 15 includes an outer sheath 20 and an inner sheath 30, and a transducer unit 40 and a shaft 50 that supports (holds) the transducer unit 40 are provided inside the sheath 15.

The outer sheath 20 is a tubular body to be inserted into a lumen of a living body. The outer sheath 20 can include, from a proximal end toward a distal end, a proximal end side tubular portion 23, a bent portion 24, and a distal end side tubular portion 25. An accommodation lumen 21 through which the proximal end communicates with the distal end is formed inside the proximal end side tubular portion 23, the bent portion 24, and the distal end side tubular portion 25.

The proximal end side tubular portion 23 is a tubular body having a substantially linear axial center. A proximal portion of the proximal end side tubular portion 23 is fixed to the first housing 60. An axial center of at least a distal portion of the proximal end side tubular portion 23 is located on a linear reference line X. The bent portion 24 is a tubular body that is located on the distal end side of the proximal end side tubular portion 23 and has a bent axial center. The distal end side tubular portion 25 is a tubular body that is located on the distal end side of the bent portion 24 and has a linear axial center. A distal portion of the distal end side tubular portion 25 is fixed to a cap 22.

A bending angle θ of the bent portion 24 is not particularly limited, and can be, for example, preferably 10° to 40°. When the bending angle θ is too small, an offset amount of a distal portion of the outer sheath 20 with respect to a proximal portion of the outer sheath 20 is small. The offset amount is a length from the reference line X to an axial center of a portion farthest in a direction perpendicular to the reference line X among portions of the outer sheath 20 on the distal end side of the bent portion 24. When the bending angle θ is too large, rotation and movement in an axial center direction of the drive shaft 50, which moves in the axial center direction while rotating in a state of being bent inside the bent portion 24, are likely to be hindered. In contrast, when the bending angle θ has an appropriate value, it is easy to set the offset amount of the outer sheath 20 to a desired value while stably maintaining the rotation and the movement in the axial center direction of the shaft 50.

A length of the distal portion, which is a length along the reference line X from a boundary between the proximal end side tubular portion 23 and the bent portion 24 to a leading distal end of the outer sheath 20, is not particularly limited, and can be, for example, preferably 20 mm to 150 mm. Therefore, it is possible to appropriately set a length of a portion of the outer sheath 20 offset on the distal end side of the bent portion 24 (a portion whose axial center is displaced from the reference line X in a direction perpendicular to the reference line X) along the reference line X so as to use the ultrasound catheter in a heart or a blood vessel having a wide lumen. In addition, when the length of the distal portion is too small, the offset amount of the distal portion of the outer sheath 20 with respect to the proximal portion of the outer sheath 20 tends to be small. When the distal portion length is too large, the offset amount of the distal portion of the outer sheath 20 with respect to the proximal portion of the outer sheath 20 tends to be large. In contrast, when the distal portion length is an appropriate length, it is easy to set the offset amount of the outer sheath 20 to a desired value.

The offset amount is not particularly limited, and can be, for example, preferably 5 mm to 30 mm. When the offset amount has an appropriate value, it is relatively easy to bring the outer sheath 20 close to a site to be observed in the heart or the blood vessel having a wide lumen.

In the outer sheath 20, the transducer unit 40, the inner sheath 30, and the shaft 50 are accommodated in the accommodation lumen 21. The transducer unit 40, the inner sheath 30, and the shaft 50 in the outer sheath 20 are movable in the accommodation lumen 21 along an axial center of the outer sheath 20. Further, the transducer unit 40 and the shaft 50 in the outer sheath 20 are rotatable inside the outer sheath 20. The outer sheath 20 is a tubular body whose proximal end is open and distal end is closed by the cap 22. The proximal portion of the proximal end side tubular portion 23 that constitutes the proximal portion of the outer sheath 20 is fixed to the first housing 60. The distal portion of the distal end side tubular portion 25 that constitutes the distal portion of the outer sheath 20 is fixed to the cap 22. The proximal portion of the outer sheath 20 may be provided with a reinforcement body such as a braided braid wire.

In the present embodiment, one bent portion 24 is provided, but the bent portion 24 may not be provided or two or more bent portions 24 may be provided.

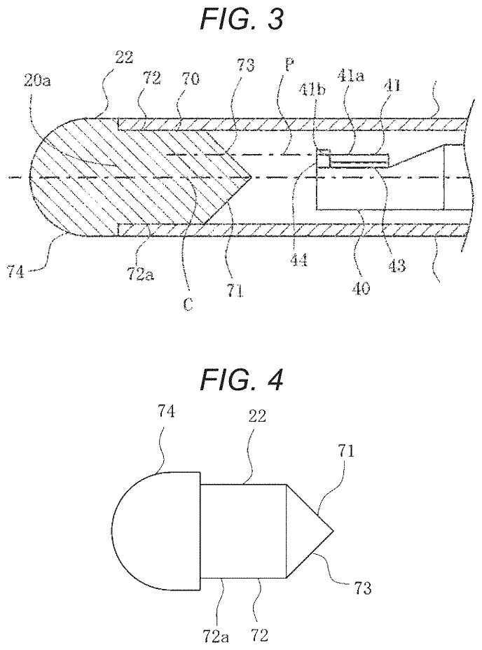

As shown in FIGS. 2 and 3, the transducer unit 40 is disposed at a distal portion of the sheath 15, and an opening portion 20a on the distal end side of the outer sheath 20 constituting the sheath 15 is sealed with the cap 22.

The transducer unit 40 transmits and receives ultrasound to and from a biological tissue in a body. The transducer unit 40 includes an ultrasound transducer 41 that transmits and receives the ultrasound, and a transducer supporting portion 42 in which the ultrasound transducer 41 is disposed and which is fixed to the shaft 50. The transducer unit 40 is movable in an axial center direction of the outer sheath 20 beyond the bent portion 24 in the accommodation lumen 21 of the outer sheath 20. In addition, the transducer unit 40 is rotatable about an axial center in the accommodation lumen 21.

In the transducer unit 40, the ultrasound transducer 41 has an ultrasound oscillation surface 41a substantially parallel to a central axis C of the sheath 15, and a distal end side surface 41b that is a side surface substantially perpendicular to the ultrasound oscillation surface 41a and faces the cap 22. The ultrasound transducer 41 may be inclined, for example, at an angle of 15° or less with respect to the central axis C of the sheath 15. The ultrasound oscillation surface 41a is disposed at a separate position radially outward from the central axis C of the sheath 15.

A back side ultrasound attenuation member 43 is provided between the transducer supporting portion 42 and a surface of the ultrasound transducer 41 opposite to the ultrasound oscillation surface 41a. In addition, a distal end side ultrasound attenuation member 44 is provided on the distal end side surface 41b of the ultrasound transducer 41 at a portion facing the cap 22. Both of the back side ultrasound attenuation member 43 and the distal end side ultrasound attenuation member 44 have a property of attenuating ultrasound oscillated from the ultrasound transducer 41 so that the ultrasound is not reflected toward the ultrasound transducer 41. Here, the “property of attenuating ultrasound” refers to a property of scattering or absorbing the ultrasound. As the back side ultrasound attenuation member 43 and the distal end side ultrasound attenuation member 44, for example, an ultrasound scattering member obtained by mixing a filler such as granular glass beads or polyethylene beads with a resin can be used, but the invention is not limited to the filler such as granular glass beads or polyethylene beads with a resin, and any material that attenuates ultrasound can be used.

The cap 22 contains an X-ray-impermeable material, and constitutes an X-ray opaque portion having an X-ray opaque property. As the X-ray-impermeable material, for example, barium sulfate, bismuth oxide, tungsten, gold, platinum, and tantalum can be used. As shown in FIGS. 2 and 4, the cap 22 includes an extension portion 70 extending from the opening portion 20a of the outer sheath 20 into the outer sheath 20, and a distal end protruding portion 74 protruding from the opening portion 20a toward the distal end side and having an outer diameter substantially the same as an outer diameter of the outer sheath 20. The extension portion 70 has a proximal portion 71 facing the ultrasound transducer 41 and an outer circumferential surface 72 facing an inner circumferential surface of the outer sheath 20. The outer circumferential surface 72 includes a contact portion 72a that is in contact with the inner circumferential surface of the outer sheath 20 in a liquid-tight manner.

The proximal portion 71 has an ultrasound scattering surface 73 that scatters ultrasound propagating from the ultrasound transducer 41 toward the proximal portion 71. The ultrasound scattering surface 73 intersects with a virtual plane P obtained by enlarging the ultrasound oscillation surface 41a in an in-plane direction and is inclined with respect to a plane perpendicular to the central axis C of the sheath 15. The ultrasound scattering surface 73 is formed on a surface of a substantially cone shape that is convex from the contact portion 72a toward the proximal end side and has a vertex disposed on the central axis C of the sheath 15. Therefore, ultrasound oscillated from the distal end side surface 41b of the ultrasound transducer 41 toward a distal end direction is reflected by the ultrasound scattering surface 73 toward a direction different from a direction in which the ultrasound transducer 41 exists. Since the ultrasound transducer 41 is disposed such that the ultrasound oscillation surface 41a is located at a separate position radially outward from the central axis C of the sheath 15, ultrasound propagating from the distal end side surface 41b of the ultrasound transducer 41 toward the cap 22 is reflected by an inclined portion of the ultrasound scattering surface 73 other than the vertex. Therefore, the ultrasound can be reliably reflected toward directions other than that of the ultrasound transducer 41.

The distal end protruding portion 74 has a dome shape that is convex toward the distal end side. Since the distal end protruding portion 74 is a distal end of the ultrasound catheter 10, a shape of the distal end protruding portion 74 is the dome shape, which can help prevent a biological tissue from being damaged.

The cap 22 contains an X-ray-impermeable material in both the extension portion 70 and the distal end protruding portion 74. Therefore, the cap 22 has an X-ray opaque property over the extension portion 70 and the distal end protruding portion 74, the contrast-enhanced portion is relatively large, and thus the X-ray opaque property can be improved.

As shown in FIG. 1, the inner sheath 30 is a tubular body whose one part on the distal end side is inserted into the outer sheath 20. The part on the distal end side of the inner sheath 30 is accommodated inside the outer sheath 20 so as to be movable along the axial center of the outer sheath 20. A proximal portion of the inner sheath 30 extends from the outer sheath 20 and the first housing 60 to the proximal end side, and is fixed to a second housing.

The inner sheath 30 rotatably accommodates the shaft 50. A distal portion of the inner sheath 30 is located on the proximal end side of the transducer unit 40 and close to the transducer unit 40. The inner sheath 30 is disposed between the inner circumferential surface of the outer sheath 20 and an outer circumferential surface of the shaft 50 to stabilize movement of the shaft 50 in a rotation direction and an axial direction.

Constituent materials for the fabrication of the outer sheath 20 and the inner sheath 30 are not particularly limited as long as the materials are flexible and have a certain degree of strength. For example, the constituent materials for the fabrication of the outer sheath 20 and the inner sheath can be a polyolefin such as polyethylene and polypropylene, polyester such as polyamide and polyethylene terephthalate, fluorine-based polymer such as polytetrafluoroethylene (PTFE) and copolymer of ethylene and tetrafluoroethylene (ETFE), polyether ether ketone (PEEK), and polyimide.

The shaft 50 transmits a rotational force and a moving force in the axial center direction, which are applied from a drive unit, to the transducer unit 40. When the shaft 50 transmits power of rotation, the transducer unit 40 rotates, and an internal structure of a tissue can be observed 360 degrees from a blood vessel or a cardiac cavity. In addition, the shaft 50 is movable along the axial center of the outer sheath 20 in the accommodation lumen 21 of the outer sheath 20.

The proximal portion of the outer sheath 20 is fixed to the housing 60 in a liquid-tight manner, and the housing 60 can include a first port 62 and a second port 64. A constituent material of the first housing 60 is not particularly limited as long as the material has a certain degree of strength. For example, the constituent material of the first housing 60 can be polycarbonate, polyamide, polysulfone, polyarylate, or methacrylate-butylene-styrene copolymer.

Ultrasound scanning by the ultrasound catheter 10 is performed by transmitting a rotational motion to the shaft 50 and rotating the transducer unit 40 fixed to a distal end of the shaft 50. Accordingly, ultrasound transmitted and received by the ultrasound transducer 41 can be used for scanning substantially in a radial direction. Further, by pulling the shaft 50 toward the proximal end side, the ultrasound transducer 41 can be moved toward the proximal end side while being rotated. Therefore, a 360° cross-sectional image of a surrounding tissue of a blood vessel or a cardiac cavity can be obtained in a scanning manner along the axial center of the outer sheath 20 to any position.

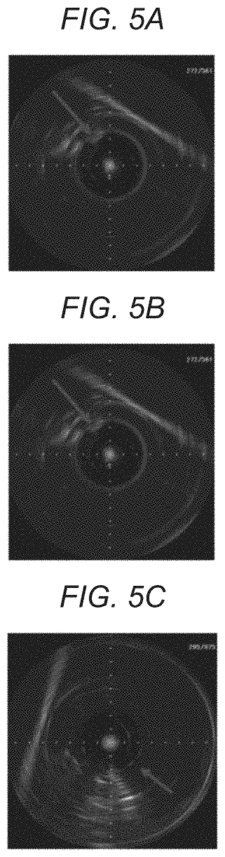

As shown in FIG. 5A, when the cap 22 having no ultrasound scattering surface 73 is attached to the opening portion 20a of the outer sheath 20 and the distal end side ultrasound attenuation member 44 is not provided on the distal end side surface 41b of the ultrasound transducer 41, ring-shaped noise indicated by an arrow is generated in an image of a biological tissue acquired by the ultrasound catheter 10. As shown in FIG. 5B, when the cap 22 having the ultrasound scattering surface 73 is attached to the opening portion 20a of the outer sheath 20, the ring-shaped noise is reduced in the image of the biological tissue acquired by the ultrasound catheter 10. As shown in FIG. 5C, by attaching the cap 22 having the ultrasound scattering surface 73 to the opening portion 20a of the outer sheath 20 and providing the distal end side ultrasound attenuation member 44 on the distal end side surface 41b of the ultrasound transducer 41, the ring-shaped noise is hardly visible in the image of the biological tissue acquired by the ultrasound catheter 10.

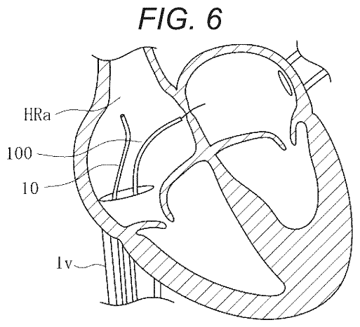

For example, as shown in FIG. 6, the ultrasound catheter 10 according to the present embodiment can be used to be inserted into a right atrium HRa from a femoral vein Iv and observe a procedure state performed by a treatment catheter 100.

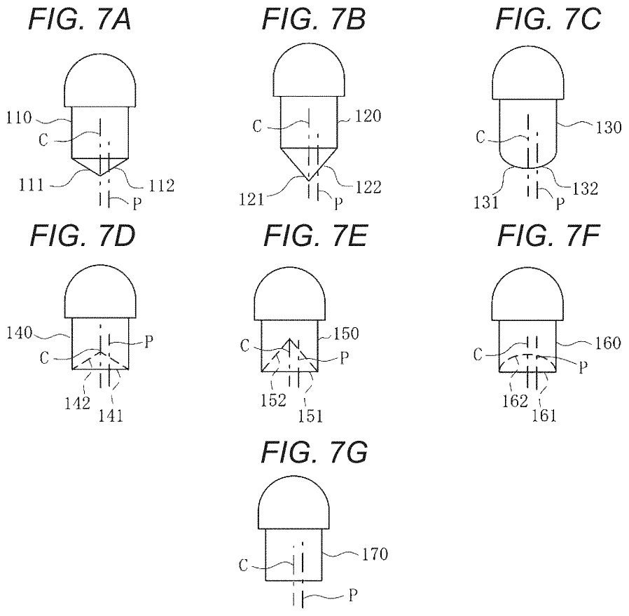

Modifications of the cap will be described. In both a cap 110 in FIG. 7A and a cap 120 in FIG. 7B, an ultrasound scattering surface 112 of a proximal portion 111 and an ultrasound scattering surface 122 of a proximal portion 121 have a substantially cone shape. The ultrasound scattering surfaces 112 and 122 are both in a state of intersecting with the virtual plane P obtained by enlarging the ultrasound oscillation surface 41a in the in-plane direction and being inclined with respect to a plane perpendicular to the central axis C of the sheath 15. The ultrasound scattering surface 112 in FIG. 7A and the ultrasound scattering surface 122 in FIG. 7B have different inclination angles with respect to a plane orthogonal to central axes of the caps 110 and 120. With the increase in the inclination angle, an effect of reducing the ring-shaped noise by scattering the ultrasound can be improved. However, when the inclination angle is too large, the cap is relatively long, and the ultrasound transducer 41 cannot be moved to the distal end side of the sheath 15. The inclination angle can be set in a range of, for example, 35° to 60°.

A cap 130 in FIG. 7C is formed such that an ultrasound scattering surface 132 of a proximal portion 131 has a dome shape that is convex toward the ultrasound transducer 41. Even in such a dome shape, the ultrasound scattering surface 132 is in a state of intersecting with the virtual plane P obtained by enlarging the ultrasound oscillation surface 41a in the in-plane direction and being inclined with respect to the plane perpendicular to the central axis C of the sheath 15, and the ultrasound from the ultrasound transducer 41 can be scattered.

Both a cap 140 in FIG. 7D and a cap 150 in FIG. 7E are formed such that an ultrasound scattering surface 142 of a proximal portion 141 and an ultrasound scattering surface 152 of a proximal portion 151 have a concave shape in a direction away from the ultrasound transducer 41, that is, a substantially cone shape in which a vertex is disposed on the central axis C of the sheath 15. Accordingly, the ultrasound scattering surfaces 142 and 152 may be concave, and the ultrasound scattering surfaces 142 and 152 are in a state of intersecting with the virtual plane P obtained by enlarging the ultrasound oscillation surface 41a in the in-plane direction and being inclined with respect to the plane perpendicular to the central axis C of the sheath 15. The ultrasound scattering surface 142 and the ultrasound scattering surface 152 have different inclination angles with respect to a plane orthogonal to central axes of the caps 140 and 150. The inclination angles can be set in a range of, for example, 35° to 60°, as when the ultrasound scattering surfaces have a convex shape.

A cap 160 in FIG. 7F is formed such that an ultrasound scattering surface 162 of a proximal portion 161 has a dome shape that is concave in a direction away from the ultrasound transducer 41. The ultrasound scattering surface 162 can scatter the ultrasound from the ultrasound transducer 41 in a state in which the ultrasound scattering surface 162 intersects with the virtual plane P obtained by enlarging the ultrasound oscillation surface 41a in the in-plane direction and is inclined with respect to the plane perpendicular to the central axis C of the sheath 15.

The following Table 1 shows generation levels of ring-shaped noise when the caps shown in FIGS. 7A to 7F each having the ultrasound scattering surface and a cap 170 shown in FIG. 7G having no ultrasound scattering surface as a comparative example were used. Accordingly, the noise levels of the caps shown in FIGS. 7A to 7F were lower than that of the cap 170 having no ultrasound scattering surface. Among them, in particular, when the ultrasound scattering surface had the substantially cone shape that is convex toward the ultrasound transducer 41, the noise level was relatively low.

Table 1

TABLE 1

| Shape | FIG. 7A | FIG. 7B | FIG. 7C | FIG. 7D | FIG. 7E | FIG. 7F | FIG. 7G |
|---|---|---|---|---|---|---|---|
| noise | low | low | medium | medium | medium | medium | high |
| level | | | | | | | |

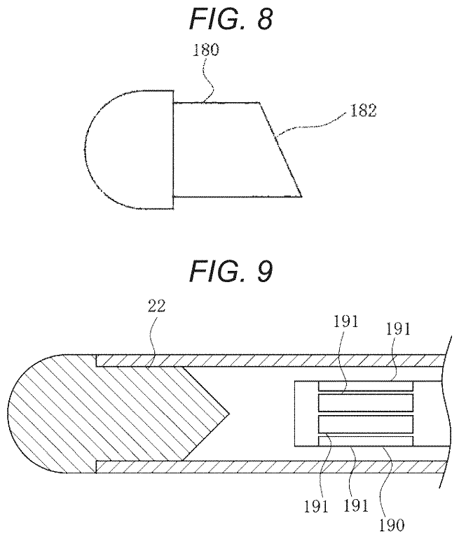

The ultrasound scattering surface may have a substantially pyramidal shape that is convex or concave toward the ultrasound transducer 41. In addition, as shown in FIG. 8, an ultrasound scattering surface 182 may be a plane inclined in one direction at a constant angle with respect to a plane orthogonal to a central axis of a cap 180.

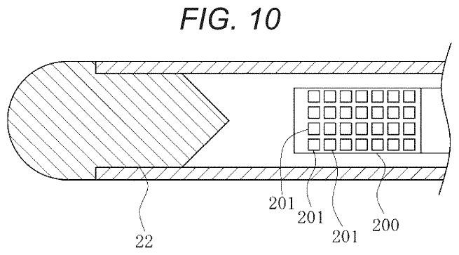

A modification of the ultrasound transducer will be described. As shown in FIG. 9, a plurality of ultrasound transducers 191 may be arranged along a circumferential direction in a transducer unit 190. The ultrasound transducers 191 can be arranged over the entire circumference of the transducer unit 190. Accordingly, a 360° cross-sectional image of a surrounding tissue of a blood vessel or a cardiac cavity can be obtained without rotating the transducer unit 190.

As shown in FIG. 10, a plurality of ultrasound transducers 201 may be arranged in an axial direction and a radial direction in a transducer unit 200. In this case, the ultrasound transducers 201 are arranged in a lattice pattern on a planar arrangement surface 200a of the transducer unit 200. Accordingly, a fan-shaped cross-sectional image of the surrounding tissue can be obtained.

As described above, the ultrasound catheter 10 according to the present embodiment includes: the elongated sheath 15 having the opening portion 20a at the distal end; the ultrasound transducer 41 accommodated in the sheath 15; the shaft 50 supporting the ultrasound transducer 41 in the sheath 15; and the cap 22 sealing the opening portion 20a of the sheath 15 and including the contrast-enhanced portion having the X-ray opaque property. The ultrasound transducer 41 includes the ultrasound oscillation surface 41a that is substantially parallel to the central axis C of the sheath 15 or is inclined, for example, at an angle of 15 degrees or less with respect to the central axis C of the sheath 15. The cap 22 includes the extension portion 70 including the proximal portion 71 extending from the opening portion 20a into the sheath 15 and facing the ultrasound transducer 41 and the outer circumferential surface 72 facing the inner circumferential surface of the sheath 15. The proximal portion 71 has the ultrasound scattering surface 73 that scatters the ultrasound propagating from the ultrasound transducer 41 toward the proximal portion 71. The outer circumferential surface 72 includes the contact portion 72a that is in contact with the inner circumferential surface of the sheath 15 in a liquid-tight manner. The ultrasound scattering surface 73 intersects with the virtual plane P obtained by enlarging the ultrasound oscillation surface 41a in the in-plane direction, and is inclined with respect to the plane perpendicular to the central axis C of the sheath 15. In the ultrasound catheter 10 configured in this way, the opening portion 20a of the sheath 15 is reliably sealed with the cap 22 having the contrast-enhanced property, and noise generated by reflection of ultrasound propagating from the ultrasound transducer 41 toward the cap 22 to an ultrasound transducer 41 side can be reduced.

The ultrasound scattering surface 73 may be formed on a surface of a substantially conical portion that is convex from the contact portion 72a toward the proximal end side and has a vertex disposed on the central axis C of the sheath 15. Accordingly, the ultrasound from the ultrasound transducer 41 can be reliably reflected toward a side other than the ultrasound transducer 41 side.

The substantially conical portion may have a substantially cone shape. Accordingly, a scattering direction of the ultrasound reflected by the ultrasound scattering surface 73 is relatively constant, and a noise reduction effect can be enhanced.

The substantially conical portion may be formed such that the ultrasound scattering surface 73 has an inclination angle of 35 degrees or larger with respect to the plane perpendicular to the central axis C of the sheath 15. Accordingly, the noise can be further reduced.

The ultrasound oscillation surface 41a may be disposed at a separate position radially outward from the central axis C of the sheath 15. Accordingly, since the vertex of the substantially conical portion is not located in the virtual plane extending from a distal surface of the ultrasound transducer 41 in parallel to the ultrasound oscillation surface 41a, the ultrasound propagating from the ultrasound transducer 41 toward the cap 22 can be more reliably scattered by the ultrasound scattering surface 73, and the noise can be reduced.

The ultrasound scattering surface 73 may be formed on a surface of a substantially conical portion that is concave in a direction away from the ultrasound transducer 41 and has a vertex disposed on the central axis C of the sheath 15. Accordingly, the ultrasound from the ultrasound transducer 41 can be reliably reflected toward a side other than the ultrasound transducer 41 side.

The cap 22 may include, at a distal end of the extension portion 70, the distal end protruding portion 74 that protrudes from the opening portion 20a toward the distal end side and has the outer diameter substantially the same as an outer diameter of the sheath 15, and the contrast-enhanced portion may be constituted by a portion including the distal end protruding portion 74 and the extension portion 70. Accordingly, a size of the contrast-enhanced portion can be increased, and the contrast-enhanced property can be improved.

The distal end protruding portion 74 may have a dome shape that is convex toward the distal end side. Accordingly, it is possible to help prevent a biological tissue from being damaged by the shape of a distal end of the catheter 10.

The ultrasound transducer 41 may include the ultrasound attenuation member 44 covering at least a portion which faces the proximal portion 71 of the cap 22 of a side surface substantially perpendicular to the ultrasound oscillation surface 41a. Accordingly, the ultrasound propagating from the ultrasound transducer 41 toward the cap 22 can be further scattered, and the noise can be further reduced.

The ultrasound transducer 41 may move substantially along the central axis C of the sheath 15 by the shaft 50. Accordingly, it is possible to acquire a cross-sectional image along a running direction of a blood vessel in a state in which the noise caused by the ultrasound reflected by the cap 22 can be reduced.

The detailed description above describes embodiments of an ultrasound catheter that acquires an image by insertion into a lumen of a heart or a blood vessel. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An ultrasound catheter comprising:
an elongated sheath having an opening portion at a distal end;
an ultrasound transducer accommodated in the sheath;
a shaft supporting the ultrasound transducer in the sheath;
a cap sealing the opening portion of the sheath and including an X-ray opaque portion having an X-ray opaque property;
the ultrasound transducer including an ultrasound oscillation surface that is substantially parallel to a central axis of the sheath or inclined at an angle of 15 degrees or less with respect to the central axis of the sheath;
the cap includes an extension portion including a proximal portion extending from the opening portion into the sheath and facing the ultrasound transducer and an outer circumferential surface facing an inner circumferential surface of the sheath, the proximal portion of the cap including an ultrasound scattering surface that scatters ultrasound directed from the ultrasound transducer toward the proximal portion, and the outer circumferential surface of the cap includes a contact portion that is in contact with the inner circumferential surface of the sheath in a liquid-tight manner; and
wherein the ultrasound scattering surface intersects with a virtual plane obtained by enlarging the ultrasound oscillation surface in an in-plane direction, and is inclined with respect to a plane perpendicular to the central axis of the sheath.

2. The ultrasound catheter according to claim 1, wherein the ultrasound scattering surface is formed on a surface of a substantially conical portion that is convex from the contact portion toward a proximal end side and has a vertex disposed on the central axis of the sheath.

3. The ultrasound catheter according to claim 2, wherein the substantially conical portion has a substantially cone shape.

4. The ultrasound catheter according to claim 2, wherein the substantially conical portion is formed such that the ultrasound scattering surface has an inclination angle of 35 degrees or larger with respect to the plane perpendicular to the central axis of the sheath.

5. The ultrasound catheter according to claim 1, wherein the ultrasound oscillation surface is disposed at a separate position radially outward from the central axis of the sheath.

6. The ultrasound catheter according to claim 1, wherein the ultrasound scattering surface is formed on a surface of a substantially conical portion that is concave in a direction away from the ultrasound transducer and has a vertex disposed on the central axis of the sheath.

7. The ultrasound catheter according to claim 1, wherein the cap includes, at a distal end of the extension portion, a distal end protruding portion that protrudes from the opening portion toward a distal end side and has an outer diameter substantially the same as an outer diameter of the sheath, and the contrast-enhanced portion is constituted by a portion including the distal end protruding portion and the extension portion.

8. The ultrasound catheter according to claim 7, wherein the distal end protruding portion has a dome shape that is convex toward the distal end side.

9. The ultrasound catheter according to claim 1, wherein the ultrasound transducer includes an ultrasound attenuation member covering at least a portion which faces the proximal portion of the cap of a side surface substantially perpendicular to the ultrasound oscillation surface.

10. The ultrasound catheter according to claim 1, wherein the ultrasound transducer moves substantially along the central axis of the sheath by the shaft.

11. An ultrasound catheter comprising:
an elongated sheath having an opening portion at a distal end;
an ultrasound transducer accommodated in the sheath;
a cap sealing the opening portion of the sheath and including an X-ray opaque portion having an X-ray opaque property; and
the cap includes an extension portion including a proximal portion extending from the opening portion into the sheath and facing the ultrasound transducer and an outer circumferential surface facing an inner circumferential surface of the sheath, the proximal portion of the cap including an ultrasound scattering surface that scatters ultrasound directed from the ultrasound transducer toward the proximal portion, and the outer circumferential surface of the cap includes a contact portion that is in contact with the inner circumferential surface of the sheath.

12. The ultrasound catheter according to claim 11, wherein the ultrasound scattering surface intersects with a virtual plane obtained by enlarging the ultrasound oscillation surface in an in-plane direction, and is inclined with respect to a plane perpendicular to the central axis of the sheath.

13. The ultrasound catheter according to claim 11, wherein the ultrasound transducer include an ultrasound oscillation surface that is substantially parallel to a central axis of the sheath.

14. The ultrasound catheter according to claim 11, wherein the ultrasound transducer include an ultrasound oscillation surface that is inclined at an angle of 15 degrees or less with respect to a central axis of the sheath.

15. The ultrasound catheter according to claim 11, wherein the ultrasound scattering surface is formed on a surface of a substantially conical portion that is convex from the contact portion toward a proximal end side and has a vertex disposed on the central axis of the sheath.

16. The ultrasound catheter according to claim 15, wherein the substantially conical portion has a substantially cone shape.

17. The ultrasound catheter according to claim 11, wherein the ultrasound scattering surface is formed on a surface of a substantially conical portion that is concave in a direction away from the ultrasound transducer and has a vertex disposed on the central axis of the sheath.

18. The ultrasound catheter according to claim 11, wherein the cap includes, at a distal end of the extension portion, a distal end protruding portion that protrudes from the opening portion toward a distal end side and has an outer diameter substantially the same as an outer diameter of the sheath, and the contrast-enhanced portion is constituted by a portion including the distal end protruding portion and the extension portion.

19. The ultrasound catheter according to claim 18, wherein the distal end protruding portion has a dome shape that is convex toward the distal end side.

20. An ultrasound catheter comprising:
an elongated sheath having an opening portion at a distal end of the sheath;
a transducer unit having a plurality of ultrasound transducers, the transducer unit being accommodated in the sheath;
a shaft supporting the transducer unit in the sheath;
a cap sealing the opening portion of the sheath and including an X-ray opaque portion having an X-ray opaque property;
the cap includes an extension portion including a proximal portion extending from the opening portion into the sheath and facing the ultrasound transducer and an outer circumferential surface facing an inner circumferential surface of the sheath, the proximal portion of the cap including an ultrasound scattering surface that scatters ultrasound directed from the ultrasound transducer toward the proximal portion, and the outer circumferential surface of the cap includes a contact portion that is in contact with the inner circumferential surface of the sheath; and
wherein the ultrasound scattering surface intersects with a virtual plane obtained by enlarging the ultrasound oscillation surface in an in-plane direction, and is inclined with respect to a plane perpendicular to the central axis of the sheath.
